(19) United States
(12) Patent Application Publication
(10) Pub. No.: US 2026/0252844 A1
(43) Pub. Date: Aug. 27, 2026

(54) SYSTEM

(71) Applicant: SoftBank Group Corp., Tokyo (JP)

(72) Inventor: Hajime YAMASHITA, Tokyo (JP)

(73) Assignee: SoftBank Group Corp., Tokyo (JP)

(21) Appl. No.: 19/541,425

(22) Filed: Feb. 17, 2026

(30) Foreign Application Priority Data
Feb. 21, 2025 (JP) 2025-027000

(51) Int. Cl. G06N 3/042; G06N 3/092

(52) U.S. Cl. CPC G06N 3/042; G06N 3/092

(57) ABSTRACT

The system according to the embodiment comprises a dialogue unit, an analysis unit, a monitoring unit, a health management unit, and a prediction unit. The dialogue unit receives an input from an elderly person. The analysis unit analyzes the input received by the dialogue unit. The monitoring unit monitors a cognitive function and a behavioral pattern of the elderly person based on information analyzed by the analysis unit. The health management unit performs individual health management based on information obtained by the monitoring unit. The prediction unit performs progression prediction of dementia based on the information obtained by the monitoring unit.

### CROSS-REFERENCE TO RELATED APPLICATION

The present application claims priority to and incorporates by reference the entire contents of Japanese Patent Application No. 2025-027000 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to efficiently monitor a cognitive function and a behavioral pattern of an elderly person, and to perform individual health management and progression prediction of dementia.

### SUMMARY OF THE INVENTION

The system according to the embodiment comprises a dialogue unit, an analysis unit, a monitoring unit, a health management unit, and a prediction unit. The dialogue unit receives an input from an elderly person. The analysis unit analyzes the input received by the dialogue unit. The monitoring unit monitors a cognitive function and a behavioral pattern of the elderly person based on information analyzed by the analysis unit. The health management unit performs individual health management based on information obtained by the monitoring unit. The prediction unit performs progression prediction of dementia based on the information obtained by the monitoring unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

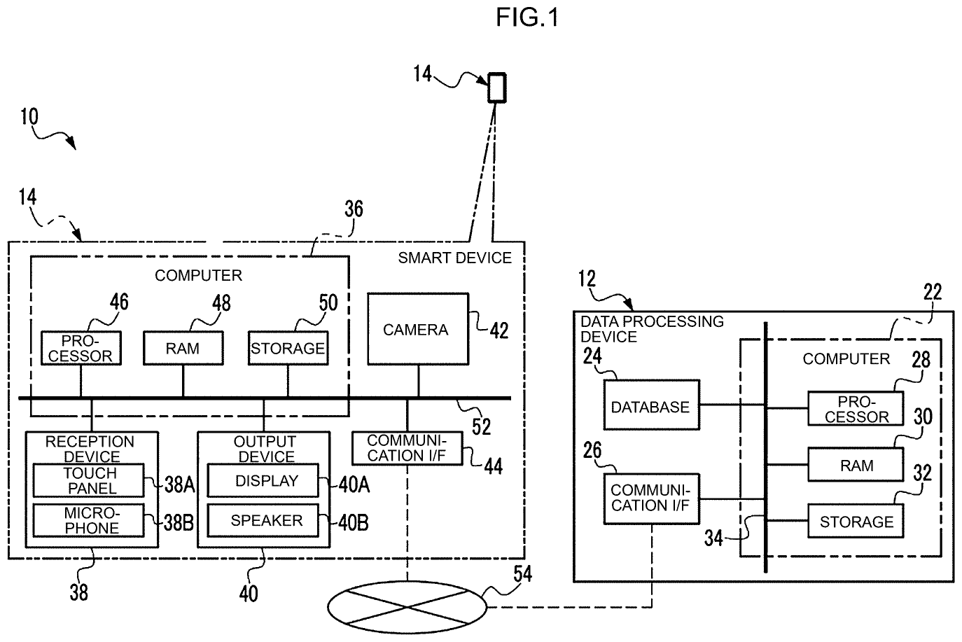

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

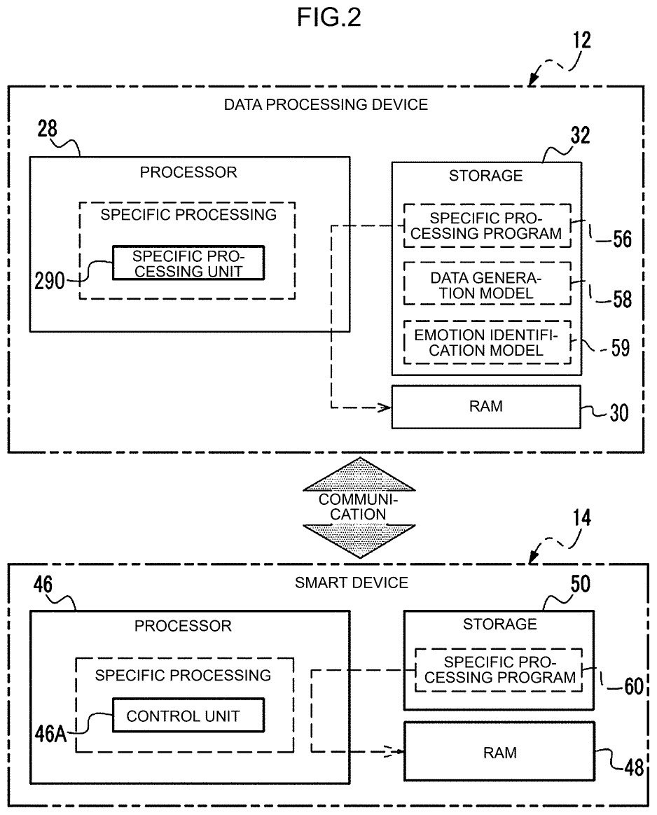

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The interactive robot system according to the embodiment of the present invention is a system for enriching the time spent at home by elderly persons with MCI (Mild Cognitive Impairment) or early-stage dementia by utilizing AI technology. This interactive robot system performs a dialogue with an elderly person, and an AI analyzes the dialogue content. Next, the AI monitors a cognitive function and a behavioral pattern of the elderly person, and performs individual health management and progression prediction of dementia. With this mechanism, the elderly person can spend time at home with peace of mind, and maintenance or improvement of the cognitive function can be expected. For example, an interactive robot performs a dialogue with the elderly person. For example, when the elderly person speaks to the interactive robot, the interactive robot makes an appropriate response. This dialogue content is analyzed by the AI. The AI analyzes the content of remarks, a tone of voice, a facial expression, and the like of the elderly person, and grasps the cognitive function and an emotional state of the elderly person. Next, the AI monitors the cognitive function and the behavioral pattern of the elderly person. For example, the interactive robot observes the behavior of the elderly person and records a change in daily life. Thereby, a change in the cognitive function of the elderly person or an abnormality in the behavioral pattern can be discovered at an early stage. Furthermore, the AI performs individual health management and progression prediction of dementia. For example, when the AI detects a decline in the cognitive function of the elderly person, it can propose an appropriate countermeasure. Also, the AI can analyze the behavioral pattern of the elderly person and predict the progression of dementia. Thereby, the health management of the elderly person is performed more effectively. Thereby, the elderly person can spend time at home with peace of mind. Through the dialogue with the interactive robot, maintenance or improvement of the cognitive function can be expected. Also, the quality of life of the elderly person is improved by the monitoring and the health management by the AI. For example, the AI discovers a decline in the cognitive function of the elderly person at an early stage and proposes an appropriate countermeasure, whereby the progression of dementia can be delayed. Also, the interactive robot observes the behavior of the elderly person and discovers an abnormality at an early stage, whereby a risk of an accident or an injury can be reduced. Thus, the interactive robot utilizing the AI technology is expected to bring about a wide range of effects such as maintenance or improvement of the cognitive function of the elderly person, health management, and progression prediction of dementia. Thereby, the interactive robot system can effectively perform maintenance or improvement of the cognitive function of the elderly person, health management, progression prediction of dementia, and the like. Specifically, the present system adopts a distributed processing architecture in which a robot terminal as an edge device and a cloud server having high computing capability are connected via a secure communication line. The present robot terminal is equipped with a multimodal sensor group such as a microphone array, a high-resolution camera, and a depth sensor, and acquires voice waveform data of the elderly person, image frame data including a facial expression, and depth map data for skeleton detection in real time. An artificial intelligence module serving as a core of the present system is composed of a multimodal deep learning model integrating a large language model based on a Transformer architecture and an image analysis model using a convolutional neural network. An input to the present artificial intelligence module is, for example, a composite dataset including a voice feature tensor obtained by converting a voice signal acquired at a sampling rate of 16 kHz into Mel-frequency cepstral coefficients, and a facial landmark coordinate vector extracted from RGB image data with a resolution of 1024×1024 pixels. The present artificial intelligence module performs a weighting operation using an attention mechanism on these input data, and outputs a score indicating a speech intention, a valence (Positive/Negative), an arousal (High/Low), and a cognitive function level (for example, a cognitive health probability normalized in a range of 0.0 to 1.0) of the elderly person. When the output score falls below a preset threshold (for example, cognitive health less than 0.4), the present system immediately transmits an alert signal to a caregiver terminal and performs control to automatically switch a speech mode of the robot to a “cognitive care mode”. With this technical configuration, unlike intermittent observation by human power in the past, it becomes possible to continuously quantitatively evaluate feature quantities that are difficult for humans to perceive, such as a minute reaction delay on a millisecond level or a trembling of a voice, 24 hours a day, 365 days a year, and accuracy of early discovery of MCI is dramatically improved.

The interactive robot system according to the embodiment includes a dialogue unit, an analysis unit, a monitoring unit, a health management unit, and a prediction unit. The dialogue unit receives an input from an elderly person. The input from the elderly person includes, for example, a voice input, a text input, and the like, but is not limited to such examples. For example, when the elderly person speaks, the dialogue unit receives the content thereof. The analysis unit analyzes the input received by the dialogue unit. The analysis includes, for example, natural language processing, emotion analysis, and the like, but is not limited to such examples. For example, the analysis unit analyzes the content of remarks, a tone of voice, a facial expression, and the like of the elderly person, and grasps a cognitive function and an emotional state of the elderly person. The monitoring unit monitors the cognitive function and a behavioral pattern of the elderly person based on information analyzed by the analysis unit. The monitoring includes, for example, recording of a behavioral pattern, a frequency of monitoring, and the like, but is not limited to such examples. For example, the monitoring unit observes the behavior of the elderly person and records a change in daily life. The health management unit performs individual health management based on information obtained by the monitoring unit. The health management includes, for example, collection of health data, a frequency of management, and the like, but is not limited to such examples. For example, when the health management unit detects a decline in the cognitive function of the elderly person, it proposes an appropriate countermeasure. The prediction unit performs progression prediction of dementia based on the information obtained by the monitoring unit. The prediction includes, for example, a prediction algorithm, data to be used, and the like, but is not limited to such examples. For example, the prediction unit analyzes the behavioral pattern of the elderly person and predicts the progression of dementia. Thereby, the interactive robot system according to the embodiment can monitor the cognitive function and the behavioral pattern of the elderly person, and perform individual health management and progression prediction of dementia. Part or all of the above-described processing in the dialogue unit may be performed using, for example, an AI, or may be performed without using an AI. For example, the dialogue unit can input the voice input of the elderly person to a generative AI and cause the generative AI to execute conversion from voice data to text data. Part or all of the above-described processing in the analysis unit may be performed using, for example, an AI, or may be performed without using an AI. For example, the analysis unit can input the content of remarks of the elderly person to a generative AI and cause the generative AI to execute analysis of the content of remarks. Part or all of the above-described processing in the monitoring unit may be performed using, for example, an AI, or may be performed without using an AI. For example, the monitoring unit can input the behavioral pattern of the elderly person to a generative AI and cause the generative AI to execute monitoring of the behavioral pattern. Part or all of the above-described processing in the health management unit may be performed using, for example, an AI, or may be performed without using an AI. For example, the health management unit can input the health data of the elderly person to a generative AI and cause the generative AI to execute a proposal for health management. Part or all of the above-described processing in the prediction unit may be performed using, for example, an AI, or may be performed without using an AI. For example, the prediction unit can input the behavioral pattern of the elderly person to a generative AI and cause the generative AI to execute progression prediction of dementia. Thereby, the interactive robot system according to the embodiment can monitor the cognitive function and the behavioral pattern of the elderly person, and perform individual health management and progression prediction of dementia. Specifically, the present dialogue unit includes an Automatic Speech Recognition (ASR) engine and a Natural Language Generation (NLG) model, and executes processing of decomposing input voice waveform data into a phoneme sequence and then converting it into a word embedding vector (Word Embedding). The present analysis unit includes an emotion analyzer in which a pre-trained language model such as BERT is fine-tuned, performs multi-class classification on an input text vector, and outputs emotion labels such as joy, sadness, anger, and anxiety, and confidence scores thereof. The present monitoring unit has a Recurrent Neural Network (RNN) or a Long Short-Term Memory (LSTM) network specialized for time-series data analysis, receives behavior log data for the past several weeks (numerical vectors such as a moving distance, a sleeping time, and a conversation amount) as an input, and calculates a deviation value of a current behavioral pattern. The present health management unit combines a rule-based inference engine based on an expert system and a recommendation engine optimized by reinforcement learning, and generates a specific health intervention plan (e.g., exercise recommendation, hydration instruction) when the calculated deviation value exceeds a predetermined threshold. The present prediction unit simulates a future cognitive function decline scenario using a Generative Adversarial Network (GAN) or a Variational Autoencoder (VAE), and outputs a predicted value of an MMSE (Mini-Mental State Examination) score several months ahead. Each of these units cooperates loosely via an API gateway and exchanges structured data in JSON format at high speed through a data bus, thereby ensuring scalability and maintainability of the entire system.

The dialogue unit is configured to estimate an emotion of the elderly person and adjust content or a tone of a dialogue based on the estimated emotion of the elderly person. For example, when the elderly person is sad, the dialogue unit speaks in a gentle tone to comfort him/her. Also, for example, when the elderly person is excited, the dialogue unit can speak in a calm tone to soothe him/her. Also, for example, when the elderly person is relaxed, the dialogue unit can provide a fun topic and enliven the dialogue. Thereby, a dialogue according to the emotion of the elderly person becomes possible. The estimation of the emotion is realized using an emotion estimation function using, for example, an emotion engine or a generative AI. The generative AI is a text generative AI (for example, LLM), a multimodal generative AI, or the like, but is not limited to such examples. Part or all of the above-described processing in the dialogue unit may be performed using, for example, an AI, or may be performed without using an AI. For example, the dialogue unit can input voice data of the elderly person to a generative AI and cause the generative AI to execute estimation of the emotion. Specifically, the present dialogue unit executes a multimodal emotion recognition model that integrates prosodic features (pitch, intensity, speech rate) extracted from a voice signal and facial Action Units extracted from a camera image. Inputs to the present model are, for example, a coefficient of variation of a fundamental frequency (F0) for each voice frame and a local feature map of a face image (for example, a vector indicating a rising degree of a mouth corner or a wrinkle between eyebrows). The present model fuses these inputs by a Cross-Attention mechanism, and quantifies and outputs an emotional state as coordinate values (Valence, Arousal) on Russell's circumplex model. Based on the output emotion coordinate values, the present dialogue unit dynamically controls parameters (speech rate, pitch average, intonation) of a speech synthesis engine (TTS). For example, when sadness (Low Valence, Low Arousal) is detected, the present dialogue unit lowers the pitch of the TTS, sets the speech rate to be slow, and applies prompt engineering indicating “empathy” to a generated response text. Thereby, not merely a fixed phrase response, but an utterance having acoustic characteristics deeply close to a psychological state of a user is generated, and a technical effect of promoting formation of rapport (relationship of trust) with the elderly person is obtained.

The dialogue unit is configured to analyze a past dialogue history of the elderly person and select an appropriate dialogue method. For example, the dialogue unit preferentially takes up a topic that the elderly person liked to talk about in the past. Also, the dialogue unit can proceed with a dialogue so as to avoid a topic that the elderly person avoided in the past, for example. Also, the dialogue unit can perform a dialogue in accordance with a time zone in which the elderly person enjoyed a dialogue in the past, for example. Thereby, an optimal dialogue method can be provided based on the past dialogue history of the elderly person. Part or all of the above-described processing in the dialogue unit may be performed using, for example, an AI, or may be performed without using an AI. For example, the dialogue unit can input the past dialogue history of the elderly person to a generative AI and cause the generative AI to execute selection of the dialogue method. Specifically, the present dialogue unit includes a vector database that vectorizes and stores all past dialogue logs, and applies Retrieval-Augmented Generation (RAG) technology. The present dialogue unit converts a current dialogue context into a query vector, and searches for and extracts a past dialogue segment having high cosine similarity in the vector database at high speed. The present dialogue unit inserts the extracted past dialogue data (a successful topic, a keyword to which the user showed a positive reaction, etc.) into a context window of a large language model as a part of a prompt. For example, when “current time: morning, user state: awake” is given as an input, the present dialogue unit refers to a history in which an utterance amount of the user increased with a topic of “gardening” in the same time zone in the past, and gives a constraint condition “generate a topic related to gardening” to a generation model. By this processing, the model can generate a personalized dialogue based on individual preference and memory of the user, and technically supports an effect of a reminiscence approach in a dementia patient.

The dialogue unit is configured to filter dialogue content based on a current health condition or an interest of the elderly person during a dialogue. For example, when the elderly person is in poor physical condition, the dialogue unit provides advice regarding health. Also, for example, when the elderly person is interested in a specific hobby, the dialogue unit can provide a topic related to the hobby. Also, for example, when the elderly person is interested in recent news, the dialogue unit can provide a topic related to the news. Thereby, a dialogue according to the current health condition or interest of the elderly person becomes possible. Part or all of the above-described processing in the dialogue unit may be performed using, for example, an AI, or may be performed without using an AI. For example, the dialogue unit can input the health condition or interest of the elderly person to a generative AI and cause the generative AI to execute filtering of the dialogue content. Specifically, the present dialogue unit holds a “user profile vector” expressing the health condition (vital signs, fatigue level) and interest (hobby, preference) of the user as a multidimensional feature vector, and performs matching processing with a generated dialogue candidate. The present dialogue unit applies a topic classification model to each of a plurality of response candidate texts output by the generative AI to generate a “topic vector”, and calculates a fitness score by inner product calculation with the user profile vector. For example, when vital data of the user indicates “high blood pressure”, the present dialogue unit executes filtering logic of subtracting a score of a topic vector related to “meal with high salt content” as a penalty, and conversely adding a score of a topic vector related to “blood pressure management”. Furthermore, the present dialogue unit includes a safety filter layer, and automatically excludes a response candidate containing a negative word or a too complex syntax that may cause anxiety or confusion to a dementia patient based on a syntax analysis result by natural language processing. Thereby, provision of information unsuitable for a mental and physical state of the user is prevented, and a safe and appropriate dialogue environment is systematically guaranteed.

The dialogue unit is configured to estimate an emotion of the elderly person and adjust a frequency of a dialogue based on the estimated emotion of the elderly person. For example, when the elderly person feels lonely, the dialogue unit increases the frequency of the dialogue. Also, for example, when the elderly person feels stress, the dialogue unit can decrease the frequency of the dialogue. Also, for example, when the elderly person is relaxed, the dialogue unit can perform a dialogue at a moderate frequency. Thereby, the frequency of the dialogue according to the emotion of the elderly person can be adjusted. The estimation of the emotion is realized using an emotion estimation function using, for example, an emotion engine or a generative AI. The generative AI is a text generative AI (for example, LLM), a multimodal generative AI, or the like, but is not limited to such examples. Part or all of the above-described processing in the dialogue unit may be performed using, for example, an AI, or may be performed without using an AI. For example, the dialogue unit can input voice data of the elderly person to a generative AI and cause the generative AI to execute estimation of the emotion. Specifically, the present dialogue unit includes a dialogue control agent implementing a Reinforcement Learning algorithm, and optimizes a policy network that determines a timing of starting a dialogue. An input (state space) to the present agent is a vector including a latest emotion score of the user, an elapsed time from a final dialogue, and a current time, and an output (action space) is a discrete action selection such as “start a dialogue” or “wait”. The present agent performs learning based on a reward function designed to receive a positive reward when the emotion score of the user improves (e.g., changes from negative to positive) after execution of a dialogue, and conversely receive a negative reward when the user shows a rejection reaction. For example, in a state where the user outputs an emotion vector indicating “loneliness”, the present agent determines that an action of increasing the dialogue frequency has a high expected reward based on past learning experience, and generates a control signal to actively speak. With this mechanism, it becomes possible to autonomously learn and execute an optimal intervention timing adapted to a dynamic emotion change of the user, instead of a fixed rule.

The dialogue unit is configured to preferentially take up a highly relevant topic based on geographical location information of the elderly person during a dialogue. For example, the dialogue unit provides weather information of a region where the elderly person lives. Also, the dialogue unit can provide event information of the region where the elderly person lives, for example. Also, the dialogue unit can provide news of the region where the elderly person lives, for example. Thereby, a highly relevant topic based on the geographical location information of the elderly person can be provided. Part or all of the above-described processing in the dialogue unit may be performed using, for example, an AI, or may be performed without using an AI. For example, the dialogue unit can input the geographical location information of the elderly person to a generative AI and cause the generative AI to execute selection of a highly relevant topic. Specifically, the present dialogue unit receives latitude and longitude data acquired from a GPS module or a Wi-Fi positioning system as an input, and identifies a specific address, regional characteristics, and peripheral facility information using a reverse geocoding API. The present dialogue unit accesses an external weather forecast API, a local news feed, and an event database using the identified regional information as a key, and acquires real-time region-related information as structured data (JSON, etc.). The present dialogue unit embeds the acquired structured data into a prompt of a natural language generation model as context information, and gives an instruction such as “generate a greeting based on the weather of the current location (XX City)”, thereby generating region-based dialogue content. Furthermore, the present dialogue unit uses geofencing technology, and performs processing of vector-searching for an episodic memory related to a specific area (content spoken at that place in the past) and including it in a topic, triggered by detection that the user has entered the specific area (e.g., a park, a shopping street). Thereby, a cognitive rehabilitation-like effect linking a place and a memory is provided through a dialogue.

The dialogue unit is configured to analyze social media activity of the elderly person during a dialogue and provide a related topic. For example, the dialogue unit provides a topic in which the elderly person showed interest on social media. Also, the dialogue unit can provide a topic related to a person whom the elderly person follows on social media, for example. Also, the dialogue unit can provide a topic related to an article shared by the elderly person on social media, for example. Thereby, a related topic based on the social media activity of the elderly person can be provided. Part or all of the above-described processing in the dialogue unit may be performed using, for example, an AI, or may be performed without using an AI. For example, the dialogue unit can input the social media activity of the elderly person to a generative AI and cause the generative AI to execute selection of a related topic. Specifically, the present dialogue unit periodically scrapes posted texts, “liked” articles, and a follow list from a social media account linked based on the user's consent, and inputs them to a natural language processing module. The present module extracts potential interest topics of the user (e.g., #travel, #grandchild, #health food) from the collected text data using a topic modeling algorithm such as Latent Dirichlet Allocation (LDA) or BERTopic, and constructs and updates an interest graph. The present dialogue unit refers to this interest graph at the time of dialogue generation, selects a topic where a currently trending topic and the user's interest intersect, and inputs it to the generative AI. For example, when it is analyzed that the user frequently reacts to posts related to “hot springs”, the present dialogue unit performs weighting so as to preferentially generate a topic related to “nearby hot spring areas” or “efficacy of hot springs”. Thereby, it becomes possible to provide a topic having high affinity for the user and stimulating a willingness to participate in a conversation, contributing to prevention of social isolation.

The analysis unit is configured to estimate an emotion of the elderly person and adjust accuracy of analysis based on the estimated emotion of the elderly person. For example, when the elderly person is relaxed, the analysis unit performs detailed analysis. Also, for example, when the elderly person feels stress, the analysis unit can perform simplified analysis. Also, for example, when the elderly person is excited, the analysis unit can perform analysis emphasizing a change in emotion. Thereby, the accuracy of analysis according to the emotion of the elderly person can be adjusted. The estimation of the emotion is realized using an emotion estimation function using, for example, an emotion engine or a generative AI. The generative AI is a text generative AI (for example, LLM), a multimodal generative AI, or the like, but is not limited to such examples. Part or all of the above-described processing in the analysis unit may be performed using, for example, an AI, or may be performed without using an AI. For example, the analysis unit can input voice data of the elderly person to a generative AI and cause the generative AI to execute estimation of the emotion. Specifically, the present analysis unit includes an adaptive inference engine that dynamically controls allocation of calculation resources, and switches a scale of a neural network model to be used according to an input emotion state vector. For example, when the user is in a “relaxed” state (stable vitals, calm voice), the present analysis unit selects a large-scale model (e.g., a Transformer model with billions of parameters) capable of high-precision analysis with a large number of parameters, and deeply analyzes a context of an utterance, a hidden intention, and a minute fluctuation of a cognitive function. On the other hand, when the user is in a “stress” state or an “excited” state, the present analysis unit switches to a quantized lightweight model (e.g., DistilBERT or MobileNet) in order to prioritize a response speed (latency), and executes a simple analysis mode that extracts only a change point of emotion immediately. By this dynamic model switching control, a balance between an optimal analysis depth and response speed according to a situation is realized within limited calculation resources, and efficiency of the entire system and user experience are improved.

The analysis unit is configured to adjust a level of detail of analysis based on importance of dialogue content during analysis. For example, the analysis unit performs detailed analysis for important dialogue content. Also, the analysis unit can perform simplified analysis for daily dialogue content, for example. Also, the analysis unit can perform particularly detailed analysis for dialogue content related to health of the elderly person, for example. Thereby, the level of detail of analysis according to the importance of the dialogue content can be adjusted. Part or all of the above-described processing in the analysis unit may be performed using, for example, an AI, or may be performed without using an AI. For example, the analysis unit can input the importance of the dialogue content to a generative AI and cause the generative AI to execute adjustment of the level of detail of analysis. Specifically, the present analysis unit applies an importance determination model (binary classifier using an Attention Mechanism) to an input dialogue text, determines whether each utterance relates to “health risk”, “urgency”, or “sign of memory impairment”, and calculates an importance score (0.0 to 1.0). For a segment in which the calculated importance score exceeds a predetermined threshold (e.g., 0.8), the present analysis unit executes a full-stack natural language understanding process including syntax analysis, semantic role labeling, and coreference resolution, structures information, and records it in a medical database. On the other hand, for a daily conversation segment such as “greeting” or “back-channeling” having a low importance score, a lightweight process that performs only simple keyword extraction is applied. By this selective analysis processing, a technical effect of significantly reducing a processing load on a server while efficiently extracting medically valuable information from a huge dialogue log is produced.

The analysis unit is configured to apply a different analysis algorithm according to a category of dialogue content during analysis. For example, the analysis unit applies a health analysis algorithm to dialogue content related to health. Also, the analysis unit can apply a hobby analysis algorithm to dialogue content related to a hobby, for example. Also, the analysis unit can apply a news analysis algorithm to dialogue content related to news, for example. Thereby, an analysis algorithm according to the category of the dialogue content can be applied. Part or all of the above-described processing in the analysis unit may be performed using, for example, an AI, or may be performed without using an AI. For example, the analysis unit can input the category of the dialogue content to a generative AI and cause the generative AI to execute application of the analysis algorithm. Specifically, the present analysis unit includes a routing module that vectorizes an input text and classifies it into a predefined category such as “health”, “hobby”, “current events”, or “life” using a multi-class classifier (for example, a neural network having a Softmax layer). The present analysis unit branches a processing pipeline based on a classification result. For example, when classified into the “health” category, the present analysis unit activates a Named Entity Recognition (NER) model linked with a medical term dictionary, and executes a specialized algorithm for identifying a symptom name, a medicine name, a body part, and the like. On the other hand, when classified into the “hobby” category, the present analysis unit applies an algorithm specialized for emotion analysis and preference extraction, and learns preference of the user. Thus, by adopting a Mixture of Experts (MoE)-like architecture that dynamically selects and applies domain-specific analysis models (Expert Models), high specialization and analysis accuracy, which are difficult to achieve with a single general-purpose model, are realized in each category.

The analysis unit is configured to estimate an emotion of the elderly person and adjust a display method of an analysis result based on the estimated emotion of the elderly person. For example, when the elderly person is nervous, the analysis unit provides a simple and highly visible display method. Also, for example, when the elderly person is relaxed, the analysis unit can provide a display method including detailed information. Also, for example, when the elderly person is in a hurry, the analysis unit can provide a display method capturing main points. Thereby, the display method of the analysis result according to the emotion of the elderly person can be adjusted. The estimation of the emotion is realized using an emotion estimation function using, for example, an emotion engine or a generative AI. The generative AI is a text generative AI (for example, LLM), a multimodal generative AI, or the like, but is not limited to such examples. Part or all of the above-described processing in the analysis unit may be performed using, for example, an AI, or may be performed without using an AI. For example, the analysis unit can input voice data of the elderly person to a generative AI and cause the generative AI to execute estimation of the emotion. Specifically, the present analysis unit cooperates with a User Interface (UI) generation engine, and dynamically rewrites screen layout description data (HTML/CSS or native UI component) based on an estimated emotion state vector. For example, when the emotion of the user indicates “nervousness” or “confusion”, the present analysis unit applies a UI template that shortens text information to be displayed to the utmost limit by a summarization algorithm, enlarges a font size, and unifies colors into calm cold colors. Conversely, when it is determined that the user is “relaxed” and has high cognitive receptivity, the present analysis unit selects a template that displays rich content including a health graph and a detailed advice sentence. This processing is an adaptive interaction control technology that estimates a cognitive load of the user from the emotion state and optimizes an information presentation amount according to the allowable load capacity, contributing to elimination of the digital divide for elderly persons.

The analysis unit is configured to determine a priority of analysis based on a time zone of a dialogue during analysis. For example, the analysis unit preferentially analyzes dialogue content in the morning. Also, the analysis unit can analyze dialogue content in the daytime next, for example. Also, the analysis unit can analyze dialogue content at night last, for example. Thereby, the priority of analysis based on the time zone of the dialogue can be determined. Part or all of the above-described processing in the analysis unit may be performed using, for example, an AI, or may be performed without using an AI. For example, the analysis unit can input the time zone of the dialogue to a generative AI and cause the generative AI to execute determination of the priority of analysis. Specifically, the present analysis unit controls an insertion order into a processing queue by referring to a timestamp included in metadata of each dialogue data by logic for determining priority incorporated in a task scheduler. Considering diurnal variation (circadian rhythm) of cognitive function fluctuation of the elderly person, the present analysis unit treats data in a time zone of “morning”, in which the cognitive function is generally clearest, as high-reliability data for baseline evaluation, assigns it to a highest-priority queue (Priority Queue), and performs immediate analysis. On the other hand, since data of “night” when fatigue tends to accumulate has a high possibility of containing noise, it is assigned to a low-priority queue for batch processing, and analysis is performed collectively in a late-night zone when a server load is low. By this scheduling algorithm dependent on the time zone, analysis delay of important biological rhythm information is prevented, and a resource usage rate of the entire system is leveled.

The analysis unit is configured to adjust an order of analysis based on relevance of dialogue content during analysis. For example, the analysis unit preferentially analyzes dialogue content related to health of the elderly person. Also, the analysis unit can analyze dialogue content related to a hobby of the elderly person next, for example. Also, the analysis unit can analyze dialogue content related to news of the elderly person last, for example. Thereby, the order of analysis based on the relevance of the dialogue content can be adjusted. Part or all of the above-described processing in the analysis unit may be performed using, for example, an AI, or may be performed without using an AI. For example, the analysis unit can input the relevance of the dialogue content to a generative AI and cause the generative AI to execute adjustment of the order of analysis. Specifically, the present analysis unit uses a causal network model that analyzes a dependency relationship between dialogue sessions, and calculates an impact degree (Impact Factor) that each dialogue topic gives to subsequent health evaluation. The present analysis unit determines an execution order of analysis tasks by applying a topological sort algorithm, and processes a “health” topic including information (for example, a complaint of poor physical condition) serving as a precondition for other analysis tasks with the highest priority as a root node. Thereby, for example, it becomes possible to perform data processing in a logically consistent order, such as performing analysis of an utterance related to “yesterday's news” (memory test) after waiting for an analysis result (symptom identification) of an utterance “I have a headache”, contributing to improvement of complex diagnostic accuracy.

The monitoring unit is configured to estimate an emotion of the elderly person and adjust a criterion for monitoring based on the estimated emotion of the elderly person. For example, when the elderly person is relaxed, the monitoring unit applies a normal monitoring criterion. Also, for example, when the elderly person feels stress, the monitoring unit can relax the monitoring criterion. Also, for example, when the elderly person is excited, the monitoring unit can make the monitoring criterion strict. Thereby, the criterion for monitoring according to the emotion of the elderly person can be adjusted. The estimation of the emotion is realized using an emotion estimation function using, for example, an emotion engine or a generative AI. The generative AI is a text generative AI (for example, LLM), a multimodal generative AI, or the like, but is not limited to such examples. Part or all of the above-described processing in the monitoring unit may be performed using, for example, an AI, or may be performed without using an AI. For example, the monitoring unit can input voice data of the elderly person to a generative AI and cause the generative AI to execute estimation of the emotion. Specifically, the present monitoring unit includes a variable threshold mechanism that dynamically scales a determination threshold in an anomaly detection algorithm (for example, One-Class SVM or Isolation Forest) according to an input emotion parameter. Considering that temporary disturbance of behavior (e.g., decrease in walking speed, disturbance of speech) is likely to occur when the user is in a “stress” state, the present monitoring unit lowers a sensitivity parameter of anomaly detection (relaxes the threshold) and suppresses unnecessary alert issuance due to false detection (False Positive). Conversely, when the user is in an “excited” state, since a risk of falling or wandering increases, the sensitivity parameter is raised (the threshold is made strict), and adjustment is made so that even a minute sign can be immediately detected. By this Bayesian inference-like threshold update processing considering the emotion context, flexible and high-precision monitoring conforming to a physiological state of a human is realized instead of mechanical monitoring.

The monitoring unit is configured to record a change in a behavioral pattern of the elderly person in real time during monitoring. For example, the monitoring unit records behavior that the elderly person performs daily, and notifies when there is a change. Also, the monitoring unit can record behavior when the elderly person starts new behavior, for example. Also, the monitoring unit can record a change when the elderly person stops performing specific behavior, for example. Thereby, the change in the behavioral pattern of the elderly person can be recorded in real time. Part or all of the above-described processing in the monitoring unit may be performed using, for example, an AI, or may be performed without using an AI. For example, the monitoring unit can input behavior data of the elderly person to a generative AI and cause the generative AI to execute recording of the change in the behavioral pattern. Specifically, the present monitoring unit is equipped with a stream processing engine that applies a sliding window method to stream data flowing in from a sensor and sequentially calculates a behavior feature quantity (moving average, variance, entropy, etc.) for each certain time frame. The present monitoring unit inputs the calculated feature quantity vector to a behavior model at a normal time (latent space distribution learned by an autoencoder), and calculates a reconstruction error in real time. The moment when this reconstruction error exceeds a statistically significant level is detected as a “change point”, and a timestamp thereof, a type of change (appearance of new behavior, disappearance of habit), and a score indicating a magnitude of the change are immediately written into a database as an event log. By this event-driven architecture, not only a gradual change day by day but also a sudden behavior modification is captured without delay, providing an opportunity for early intervention.

The monitoring unit is configured to improve accuracy of monitoring based on a change in a living environment of the elderly person during monitoring. For example, when the elderly person moves house, the monitoring unit adjusts the accuracy of monitoring based on a new living environment. Also, for example, when the elderly person introduces a new home appliance, the monitoring unit can reflect a usage situation thereof in monitoring. Also, for example, when the elderly person starts a new hobby, the monitoring unit can adjust the accuracy of monitoring based on the hobby. Thereby, the accuracy of monitoring considering the change in the living environment of the elderly person can be improved. Part or all of the above-described processing in the monitoring unit may be performed using, for example, an AI, or may be performed without using an AI. For example, the monitoring unit can input living environment data of the elderly person to a generative AI and cause the generative AI to execute improvement of the accuracy of monitoring. Specifically, the present monitoring unit detects an update of environment map data generated by a SLAM (Simultaneous Localization and Mapping) function of the robot, and recognizes a layout change of a living space or installation of a new object. When an environmental change is detected, the present monitoring unit re-learns (fine-tunes) a part of a behavior recognition model using transfer learning. For example, when a movement route changes due to new furniture arrangement, a definition of “abnormal movement trajectory” learned in an old environment is corrected according to a new environment. Also, when an IoT device such as a smart home appliance is newly added, log data from the device is integrated into a monitoring model as a new input dimension, and a weight of a multimodal input is re-optimized. Thereby, obsolescence (concept drift) of the model due to the environmental change is prevented, and high-precision monitoring adapted to the latest living environment is always maintained.

The monitoring unit is configured to estimate an emotion of the elderly person and adjust a display method of a monitoring result based on the estimated emotion of the elderly person. For example, when the elderly person is nervous, the monitoring unit provides a simple and highly visible display method. Also, for example, when the elderly person is relaxed, the monitoring unit can provide a display method including detailed information. Also, for example, when the elderly person is in a hurry, the monitoring unit can provide a display method capturing main points. Thereby, the display method of the monitoring result according to the emotion of the elderly person can be adjusted. The estimation of the emotion is realized using an emotion estimation function using, for example, an emotion engine or a generative AI. The generative AI is a text generative AI (for example, LLM), a multimodal generative AI, or the like, but is not limited to such examples. Part or all of the above-described processing in the monitoring unit may be performed using, for example, an AI, or may be performed without using an AI. For example, the monitoring unit can input voice data of the elderly person to a generative AI and cause the generative AI to execute estimation of the emotion. Specifically, the present monitoring unit executes logic for selecting a template using a current emotion state of a user as an input variable when generating a dashboard for feedback to the user (the elderly person himself/herself or a family member). When it is estimated that the user feels “anxiety” or “nervousness”, the present monitoring unit hides a complex graph or numerical data, and selects a “simple mode” that displays only a plain natural language summary giving a sense of security such as “You are sleeping well today” and a positive icon. On the other hand, when it is estimated that the user is “relaxed” and capable of analytical thinking, the present monitoring unit selects a “detailed mode” that comprehensively displays detailed data such as a step count transition, a sleep depth graph, and a conversation amount heat map. By this emotion-adaptive UI generation, checking of the monitoring result itself is prevented from becoming a stress factor for the user, and continuous use is promoted.

The monitoring unit is configured to perform monitoring in consideration of a geographical distribution of the elderly person during monitoring. For example, the monitoring unit performs monitoring in consideration of characteristics of a region where the elderly person lives. Also, the monitoring unit can perform monitoring in consideration of a place where the elderly person frequently visits, for example. Also, the monitoring unit can perform monitoring in consideration of characteristics of a travel destination when the elderly person is traveling, for example. Thereby, monitoring considering the geographical distribution of the elderly person can be performed. Part or all of the above-described processing in the monitoring unit may be performed using, for example, an AI, or may be performed without using an AI. For example, the monitoring unit can input geographical distribution data of the elderly person to a generative AI and cause the generative AI to execute the monitoring. Specifically, the present monitoring unit clusters movement trajectory data obtained from a GPS log (for example, DBSCAN algorithm), and automatically identifies “Points of Interest (POI)” such as a home, a supermarket, and a hospital. The present monitoring unit evaluates a staying time and a movement frequency at each POI against regional characteristics (amount of slopes, traffic volume, security data). For example, a decrease in walking speed in a flat area is distinguished from a decrease in walking speed in an area with many slopes, and physical function is evaluated after adding terrain data as a correction term. Also, in an unlearned area such as a travel destination, a threshold for anomaly detection is temporarily relaxed, and logic is adjusted so that a behavioral change due to an environmental factor is not erroneously determined as a cognitive function decline. By monitoring incorporating this spatial statistical analysis, a risk of misdiagnosis due to uniform evaluation ignoring a geographical context is reduced.

The monitoring unit can improve accuracy of monitoring by referring to related literature of the elderly person during monitoring. The monitoring unit performs monitoring by referring to, for example, the latest research on a health condition of the elderly person. In addition, the monitoring unit can perform monitoring by referring to, for example, literature on a behavioral pattern of the elderly person. Furthermore, the monitoring unit can perform monitoring by referring to, for example, literature on a cognitive function of the elderly person. Thereby, the accuracy of monitoring referring to the related literature of the elderly person can be improved. Part or all of the above-described processing in the monitoring unit may be performed using, for example, AI, or may be performed without using AI. For example, the monitoring unit can input related literature data of the elderly person into generative AI and cause the generative AI to execute improvement of the accuracy of monitoring. Specifically, the monitoring unit has a function of linking with a medical literature database such as PubMed via an API, extracting findings (entities and relationships) regarding “early signs” and “risk factors” from the latest dementia research papers using natural language processing, and automatically updating a knowledge graph inside the system. The monitoring unit infers which risk factor on the knowledge graph observed behavioral data corresponds to, and performs weighting based on medical evidence. For example, when it is reported in the latest literature that “variability in stride length” is a strong indicator of MCI, the monitoring unit automatically increases a weight of a feature amount of “stride variance” in a gait analysis model. This literature-driven model update mechanism makes it possible to maintain state-of-the-art monitoring accuracy that always reflects the latest medical findings.

The health management unit can estimate an emotion of the elderly person and adjust a method of health management based on the estimated emotion of the elderly person. For example, when the elderly person is relaxed, the health management unit applies a normal health management method. In addition, when the elderly person feels stress, the health management unit can propose a health management method for stress reduction. Furthermore, when the elderly person is excited, the health management unit can propose a health management method for relaxation. Thereby, the method of health management according to the emotion of the elderly person can be adjusted. Estimation of the emotion is realized using an emotion estimation function using, for example, an emotion engine or generative AI. The generative AI is text generative AI (e.g., LLM), multimodal generative AI, or the like, but is not limited to such examples. Part or all of the above-described processing in the health management unit may be performed using, for example, AI, or may be performed without using AI. For example, the health management unit can input voice data of the elderly person into generative AI and cause the generative AI to execute estimation of the emotion. Specifically, the health management unit determines a policy for maximizing the user's long-term health maintenance (Reward) using a reinforcement learning model (such as DQN) in which a user's emotion state is a state variable (State) and a health intervention (advice, exercise proposal, music playback, etc.) is an action variable (Action). When it is estimated that the user is in a “stress” state, the health management unit avoids a proposal with high exercise load and selects an intervention plan that makes the parasympathetic nerve dominant, such as deep breathing, a meditation guide, or playback of music with a sedative effect. Conversely, in the case of an “excited” state, control is performed to induce arousal of emotion (Arousal) to an appropriate range, such as proposing a cognitive game for diverting attention. This emotion feedback loop realizes comprehensive healthcare management including mental stability, not limited to mere physical health management.

The health management unit can select an optimal health management method by referring to past health data of the elderly person during health management. The health management unit selects a health management method by referring to, for example, a past health checkup result of the elderly person. In addition, the health management unit can select a health management method by referring to, for example, a past medical history of the elderly person. Furthermore, the health management unit can select a health management method by referring to, for example, a past exercise history of the elderly person. Thereby, the optimal health management method based on the past health data of the elderly person can be selected. Part or all of the above-described processing in the health management unit may be performed using, for example, AI, or may be performed without using AI. For example, the health management unit can input the past health data of the elderly person into generative AI and cause the generative AI to execute selection of the health management method. Specifically, the health management unit reads long-term time-series data (blood pressure, body weight, blood glucose level, medication history, etc.) accumulated as a personal health record (PHR) of the user, and models an individual health trend using regression analysis or an LSTM network. Based on this trend model, the health management unit determines whether a current vital value deviates from an individual reference range, and if it deviates, searches for and recommends an intervention method (e.g., a specific exercise menu or dietary restriction) that was effective in the past from history data. For example, if there is a history where blood pressure was stabilized by “walking” in the past, “walking” is preferentially proposed when a blood pressure rising tendency is observed. In this way, a data-driven approach of precision medicine based on individual physical characteristics and history, rather than general theory, is applied.

The health management unit can customize a means for health management based on a current living situation of the elderly person during health management. For example, when the elderly person lives alone, the health management unit proposes a regular health check. In addition, when the elderly person lives with a family, the health management unit can propose a method of performing health management in cooperation with the family. Furthermore, when the elderly person uses a day service, the health management unit can propose a health management method tailored to the service. Thereby, the means for health management based on the current living situation of the elderly person can be customized. Part or all of the above-described processing in the health management unit may be performed using, for example, AI, or may be performed without using AI. For example, the health management unit can input living situation data of the elderly person into generative AI and cause the generative AI to execute customization of the health management means. Specifically, the health management unit generates a “living context vector” structuring user attribute information (living alone, living together, care level) and schedule information (day service use date, home visit care schedule), and inputs it into a generation model of a health management program. For a user living alone, the health management unit applies a setting that increases a frequency of active medication confirmation and hydration alerts by a robot, while for a user having a cohabiting family, the health management unit creates a care support report for the family and generates a plan that encourages care via the family. Also, on a day service use date, optimization considering a life rhythm and a social support situation is performed, such as refraining from proposing exercise at home in anticipation of an activity amount at a facility, thereby preventing over-interference or lack of support.

The health management unit can estimate an emotion of the elderly person and determine a priority of health management based on the estimated emotion of the elderly person. For example, when the elderly person feels stress, the health management unit gives top priority to stress reduction. In addition, when the elderly person is relaxed, the health management unit can prioritize normal health management. Furthermore, when the elderly person is excited, the health management unit can prioritize health management for relaxation. Thereby, the priority of health management according to the emotion of the elderly person can be determined. Estimation of the emotion is realized using an emotion estimation function using, for example, an emotion engine or generative AI. The generative AI is text generative AI (e.g., LLM), multimodal generative AI, or the like, but is not limited to such examples. Part or all of the above-described processing in the health management unit may be performed using, for example, AI, or may be performed without using AI. For example, the health management unit can input voice data of the elderly person into generative AI and cause the generative AI to execute estimation of the emotion. Specifically, the health management unit executes a multi-objective optimization algorithm that scores urgency and importance for each of a plurality of health issues (lack of exercise, nutrition management, mental care, etc.) and further performs weighting according to a current emotion state. When the user is in a strong “stress” or “depression” state, the health management unit performs triage to temporarily lower a priority of a physical health issue (e.g., salt reduction) and maximize a priority of mental care (e.g., active listening, music therapy). This is an implementation of a clinical psychological approach as an algorithm, which avoids a risk that lifestyle guidance becomes counterproductive in a mentally unstable state and gives top priority to ensuring mental stability first.

The health management unit can select an optimal health management method in consideration of geographical location information of the elderly person during health management. The health management unit selects a health management method based on, for example, information on medical facilities in an area where the elderly person lives. In addition, the health management unit can select a health management method in consideration of, for example, a climate of the area where the elderly person lives. Furthermore, the health management unit can select a health management method in consideration of, for example, a living environment of the area where the elderly person lives. Thereby, the optimal health management method based on the geographical location information of the elderly person can be selected. Part or all of the above-described processing in the health management unit may be performed using, for example, AI, or may be performed without using AI. For example, the health management unit can input the geographical location information of the elderly person into generative AI and cause the generative AI to execute selection of the health management method. Specifically, the health management unit cooperates with GIS (Geographic Information System) data and refers to a medical resource map (distribution of hospitals, pharmacies, parks, gyms) around the user's residence. When a symptom with high urgency is suspected, the health management unit searches for a medical institution reachable in the shortest time from a current location and proposes contact information or a means of transportation thereof. In addition, the health management unit incorporates “meteoropathy” countermeasure logic that acquires regional weather data (temperature, humidity, atmospheric pressure) in real time and warns to refrain from going out on days with high heatstroke risk or encourages rest in advance when a headache due to a drop in atmospheric pressure is predicted. Thereby, specific and actionable health advice tailored to environmental factors is provided.

The health management unit can analyze social media activity of the elderly person and propose a means for health management during health management. The health management unit proposes a health management method based on, for example, health information in which the elderly person showed interest on social media. In addition, the health management unit can propose a health management method based on, for example, advice of a health expert followed by the elderly person on social media. Furthermore, the health management unit can propose a health management method based on, for example, a health article shared by the elderly person on social media. Thereby, the means for health management based on the social media activity of the elderly person can be proposed. Part or all of the above-described processing in the health management unit may be performed using, for example, AI, or may be performed without using AI. For example, the health management unit can input the social media activity of the elderly person into generative AI and cause the generative AI to execute proposal of the health management means. Specifically, the health management unit analyzes activity logs (browsing history, shares, comments) on SNS of the user and identifies a health method or a diet method in which the user is interested. The health management unit verifies the presence or absence of medical basis (evidence) for the identified health method by collating it with a reliability database. If the user is devoted to a health method with poor scientific basis, the health management unit does not deny it outright, but proposes a more medically correct alternative (e.g., if interested in “XX diet”, a balanced meal menu having a similar effect) related to the user's interest by the generative AI. Thereby, intervention based on Nudge theory is performed to prevent health damage due to misinformation while utilizing the user's spontaneous interest.

The prediction unit can estimate an emotion of the elderly person and adjust a method of progression prediction of dementia based on the estimated emotion of the elderly person. For example, when the elderly person is relaxed, the prediction unit applies a normal prediction method. In addition, when the elderly person feels stress, the prediction unit can apply a prediction method considering an influence of stress. Furthermore, when the elderly person is excited, the prediction unit can apply a prediction method emphasizing a change in emotion. Thereby, the method of progression prediction of dementia according to the emotion of the elderly person can be adjusted. Estimation of the emotion is realized using an emotion estimation function using, for example, an emotion engine or generative AI. The generative AI is text generative AI (e.g., LLM), multimodal generative AI, or the like, but is not limited to such examples. Part or all of the above-described processing in the prediction unit may be performed using, for example, AI, or may be performed without using AI. For example, the prediction unit can input voice data of the elderly person into generative AI and cause the generative AI to execute estimation of the emotion. Specifically, the prediction unit adds an emotion state vector (stress level, concentration level) at the time of test execution as an input feature amount to a dementia progression prediction model (e.g., a Transformer model handling time-series data), in addition to a score of a cognitive function test. The prediction unit learns that the cognitive function score tends to temporarily decrease when the user is under strong stress, and performs processing of estimating true cognitive ability (baseline) by applying a score decrease due to a stress factor as a correction coefficient in prediction calculation. This realizes prognosis prediction that accurately extracts only long-term pathological progression by removing noise due to temporary emotional fluctuation.

The prediction unit can select an optimal prediction method by referring to past cognitive function data of the elderly person during prediction. The prediction unit selects a prediction method by referring to, for example, a past cognitive function test result of the elderly person. In addition, the prediction unit can select a prediction method by referring to, for example, a past change in cognitive function of the elderly person. Furthermore, the prediction unit can select a prediction method by referring to, for example, a medical record regarding a past cognitive function of the elderly person. Thereby, the optimal prediction method based on the past cognitive function data of the elderly person can be selected. Part or all of the above-described processing in the prediction unit may be performed using, for example, AI, or may be performed without using AI. For example, the prediction unit can input the past cognitive function data of the elderly person into generative AI and cause the generative AI to execute selection of the prediction method. Specifically, the prediction unit analyzes score transition data of MMSE (Mini-Mental State Examination) or HDS-R (Hasegawa Dementia Scale-Revised) for the past several years, and classifies a progression pattern (rapid progression type, slow progression type, stepwise progression type). The prediction unit selects an optimal prediction algorithm according to the classified progression pattern. For example, a statistical time-series model such as an ARIMA model is applied to the slow progression type that decreases linearly, and a deep learning model such as LSTM or GRU is applied to the rapid progression type that shows non-linear and rapid change. This adaptive model selection provides high-precision future prediction consistent with pathological progression characteristics of an individual patient.

The prediction unit can customize a means for progression prediction of dementia based on a current living situation of the elderly person during prediction. For example, when the elderly person lives alone, the prediction unit proposes a regular cognitive function check. In addition, when the elderly person lives with a family, the prediction unit can propose a method of performing a cognitive function check in cooperation with the family. Furthermore, when the elderly person uses a day service, the prediction unit can propose a cognitive function check method tailored to the service. Thereby, the means for progression prediction of dementia based on the current living situation of the elderly person can be customized. Part or all of the above-described processing in the prediction unit may be performed using, for example, AI, or may be performed without using AI. For example, the prediction unit can input living situation data of the elderly person into generative AI and cause the generative AI to execute customization of the means for progression prediction of dementia. Specifically, the prediction unit uses a multivariate analysis model incorporating living environment factors (frequency of social interaction, presence or absence of intellectual activity, presence or absence of care support) as explanatory variables. For a user living alone with little social interaction, the prediction unit estimates a risk coefficient of cognitive decline to be high, sets a prediction period short (e.g., prediction every month), and executes a simulation encouraging frequent checks. On the other hand, for a user receiving sufficient stimulation at a day service or the like, prediction is performed from a longer-term perspective in consideration of a protective effect (Cognitive Reserve) due to environmental factors. Thereby, an influence of the living environment on progression of the disease is quantitatively incorporated into evaluation.

The prediction unit can estimate an emotion of the elderly person and determine a priority of progression prediction of dementia based on the estimated emotion of the elderly person. For example, when the elderly person feels stress, the prediction unit gives top priority to stress reduction. In addition, when the elderly person is relaxed, the prediction unit can prioritize normal prediction. Furthermore, when the elderly person is excited, the prediction unit can prioritize prediction for relaxation. Thereby, the priority of progression prediction of dementia according to the emotion of the elderly person can be determined. Estimation of the emotion is realized using an emotion estimation function using, for example, an emotion engine or generative AI. The generative AI is text generative AI (e.g., LLM), multimodal generative AI, or the like, but is not limited to such examples. Part or all of the above-described processing in the prediction unit may be performed using, for example, AI, or may be performed without using AI. For example, the prediction unit can input voice data of the elderly person into generative AI and cause the generative AI to execute estimation of the emotion. Specifically, the prediction unit sets a user's emotion state as a trigger condition in a scheduler that controls execution timing of a prediction task. When the user is in a “depressed state” or “high stress”, the prediction unit postpones execution of a burdensome prediction task such as a cognitive function test, and instead raises a priority of a task for predicting a risk of deterioration of mental health. This is to manage a risk of comorbidity of dementia and depression, and the system operates autonomously so as to minimize a psychological influence of a prediction act itself on the user, not just predicting a cognitive function.

The prediction unit can select an optimal prediction method in consideration of geographical location information of the elderly person during prediction. The prediction unit selects a prediction method based on, for example, information on medical facilities in an area where the elderly person lives. In addition, the prediction unit can select a prediction method in consideration of, for example, a climate of the area where the elderly person lives. Furthermore, the prediction unit can select a prediction method in consideration of, for example, a living environment of the area where the elderly person lives. Thereby, the optimal prediction method based on the geographical location information of the elderly person can be selected. Part or all of the above-described processing in the prediction unit may be performed using, for example, AI, or may be performed without using AI. For example, the prediction unit can input the geographical location information of the elderly person into generative AI and cause the generative AI to execute selection of the prediction method. Specifically, the prediction unit refers to regional epidemiological data (prevalence of dementia by region, tendency of lifestyle-related diseases) as background data, and adjusts a prior distribution of a prediction model. For example, for a user residing in an area with a high risk of cerebrovascular disease in a cold region, a prediction model specialized for a progression pattern of vascular dementia is selected, and changes in atmospheric pressure and temperature are incorporated as important predictors. In addition, for a user residing in a medically underserved area, a prediction scenario considering a risk of aggravation due to difficulty in hospital visits is generated. This enables more realistic and highly accurate prognosis prediction considering environmental factors derived from regional characteristics.

The prediction unit can analyze social media activity of the elderly person and propose a means for progression prediction of dementia during prediction. The prediction unit proposes a prediction method based on, for example, health information in which the elderly person showed interest on social media. In addition, the prediction unit can propose a prediction method based on, for example, advice of a health expert followed by the elderly person on social media. Furthermore, the prediction unit can propose a prediction method based on, for example, a health article shared by the elderly person on social media. Thereby, the means for progression prediction of dementia based on the social media activity of the elderly person can be proposed. Part or all of the above-described processing in the prediction unit may be performed using, for example, AI, or may be performed without using AI. For example, the prediction unit can input the social media activity of the elderly person into generative AI and cause the generative AI to execute proposal of the means for progression prediction of dementia. Specifically, the prediction unit performs linguistic analysis on posted texts on SNS to quantify vocabulary diversity (Type-Token Ratio), grammatical complexity, and temporal change in posting frequency. The prediction unit detects a decreasing tendency of these linguistic feature amounts (e.g., increased use of pronouns or shortening of sentences) as an early marker of cognitive decline, and constructs a multimodal prediction model that predicts a future progression risk by integrating it with a result of a conventional cognitive function test. Furthermore, by incorporating an effect of a preventive method (e.g., brain training app, supplement) in which the user shows interest on SNS into prediction simulation and visualizing and proposing “progression suppression effect if this preventive method is continued”, the user's behavioral change is encouraged.

The system according to the embodiment is not limited to the above-described examples, and various modifications are possible, for example, as follows. Specifically, an architecture of the present system is designed flexibly without depending on a specific hardware configuration, and can be changed to not only a cloud-based centralized processing type but also a distributed processing type utilizing edge computing, or a hybrid configuration combining them. In addition, each functional module (dialogue unit, analysis unit, monitoring unit, etc.) is containerized based on a microservice architecture, and can be individually scaled, updated, or replaced using an orchestration tool such as Kubernetes. Furthermore, the present system publishes a standardized API (RESTful API or gRPC), and has scalability capable of easily linking with a third-party healthcare device (smart watch, sleep meter) or an external medical information system (electronic medical record) to perform function expansion.

The dialogue unit can customize dialogue content based on a hobby or an interest of the elderly person. For example, when the elderly person is interested in gardening, the dialogue unit provides a topic regarding gardening. In addition, when the elderly person likes music, the dialogue unit can provide a topic regarding music. Furthermore, when the elderly person is interested in traveling, the dialogue unit can provide a topic regarding traveling. This enables a dialogue according to the hobby or interest of the elderly person. Specifically, the dialogue unit refers to an ontology-based knowledge base that manages user hobbies and preferences as a hierarchical category structure. The dialogue unit maps a keyword (e.g., “rose”, “fertilizer”) extracted from a user's utterance to the knowledge base, infers a related subordinate concept or related concept (e.g., “pruning time”, “watering frequency”), and develops a topic. Furthermore, the dialogue unit cooperates with an API of an external content provider (music distribution service, travel information site) to acquire specific content (e.g., a hit song of a specific era, a seasonal tourist spot) matching the user's preference in real time and incorporate it into the dialogue, thereby functioning as an information concierge that provides specific experiences and information, not just a conversation.

The dialogue unit can estimate an emotion of the elderly person and adjust content or a tone of a dialogue based on the estimated emotion of the elderly person. For example, when the elderly person is sad, the dialogue unit speaks in a comforting gentle tone. In addition, when the elderly person is excited, the dialogue unit can speak in a calm tone to soothe. Furthermore, when the elderly person is relaxed, the dialogue unit can provide a fun topic to enliven the dialogue. This enables a dialogue according to the emotion of the elderly person. Specifically, the dialogue unit uses a Neural Vocoder trained with an emotion-tagged speech corpus in a text-to-speech (TTS) system. The dialogue unit receives an estimated emotion label (e.g., Sadness, Excitement) and an intensity parameter as inputs, and finely controls a spectral envelope and a fundamental frequency pattern of a generated speech waveform. For example, for “sadness”, prosody generation is performed to emphasize a falling tone at the end of a sentence and take a longer pause, and for “excitement”, a sedative effect is aimed at by suppressing high-frequency components and speaking in a constant rhythm. In this way, by optimizing not only linguistic information (text) but also non-linguistic information (paralanguage) according to the emotion, a more human-like and empathetic dialogue interface is realized.

The dialogue unit can analyze a past dialogue history of the elderly person and select an appropriate dialogue method. For example, the dialogue unit preferentially takes up a topic that the elderly person liked to talk about in the past. In addition, the dialogue unit can proceed with a dialogue so as to avoid a topic that the elderly person avoided in the past. Furthermore, the dialogue unit can perform a dialogue in accordance with a time zone in which the elderly person enjoyed a dialogue in the past. Thereby, the optimal dialogue method based on the past dialogue history of the elderly person can be provided. Specifically, the dialogue unit accumulates an “engagement score” (degree of excitement of dialogue calculated from the number of utterances, response speed, and emotional valence) for each past dialogue session in a database. The dialogue unit uses a machine learning model (e.g., decision tree or random forest) to analyze which topic category, which time zone, and which way of speaking (honorifics, casual speech, etc.) bring the highest engagement score, and constructs a “dialogue strategy model” for each user. At the next dialogue, the most successful topic and style are selected based on this model. This reduces trial-and-error dialogues and makes it possible to provide a comfortable dialogue experience for the user from the beginning.

The dialogue unit can filter dialogue content based on a current health condition or an interest of the elderly person during a dialogue. For example, when the elderly person is in poor physical condition, the dialogue unit provides advice regarding health. In addition, when the elderly person is interested in a specific hobby, the dialogue unit can provide a topic regarding the hobby. Furthermore, when the elderly person is interested in recent news, the dialogue unit can provide a topic regarding the news. This enables a dialogue according to the current health condition or interest of the elderly person. Specifically, the dialogue unit includes a context-aware filtering engine that integrates real-time vital sensor data (heart rate, body temperature) and the latest news feed or trend information. When the vital data indicates an abnormal value, the engine blocks all entertainment-related topics (hobbies, news) and switches to an “emergency mode” that passes only a dialogue flow regarding medical interview or safety confirmation. On the other hand, when the vital is normal, similarity calculation between a user's interest vector and a news article vector is performed, and only an article exceeding a threshold is listed as a topic candidate. This dynamic filtering realizes appropriate and safe information provision according to the situation.

The dialogue unit can estimate an emotion of the elderly person and adjust a frequency of a dialogue based on the estimated emotion of the elderly person. For example, when the elderly person feels lonely, the dialogue unit increases the frequency of a dialogue. In addition, when the elderly person feels stress, the dialogue unit can decrease the frequency of a dialogue. Furthermore, when the elderly person is relaxed, the dialogue unit can perform a dialogue at a moderate frequency. Thereby, the frequency of a dialogue according to the emotion of the elderly person can be adjusted. Specifically, the dialogue unit models a time-series change in the user's emotion state as a Markov Decision Process (MDP) and optimizes the dialogue frequency (action). The dialogue unit learns that in a state where “loneliness” is high, a “call” action at short intervals yields a high reward (reduction of loneliness), and executes frequent interaction. On the other hand, in a state where “stress” is high, since the dialogue itself becomes a burden, it is determined that taking no action (No-Op) is the optimal strategy, and a silent mode is maintained. This frequency control based on a mathematical model realizes robot intervention that maintains a comfortable distance for the user.

The dialogue unit can preferentially take up a highly relevant topic based on geographical location information of the elderly person during a dialogue. For example, the dialogue unit provides weather information of an area where the elderly person lives. In addition, the dialogue unit can provide event information of the area where the elderly person lives. Furthermore, the dialogue unit can provide news of the area where the elderly person lives. Thereby, the highly relevant topic based on the geographical location information of the elderly person can be provided. Specifically, the dialogue unit cooperates with a location-based information delivery (LBS: Location-Based Services) platform to collect “hyper-local information” within a radius of several kilometers centered on the user's current location. The dialogue unit preferentially selects information closely related to daily life, such as “bargain information of a neighborhood supermarket” or “circle activity at a public hall”, as a topic, not just a weather forecast. Furthermore, by comparing with a history of places visited by the user in the past and generating a reminder-like dialogue linking memory and place, such as “Cherry blossoms are blooming in that park you went to before”, improvement of motivation for going out and stimulation of cognitive function are aimed at.

The dialogue unit can analyze social media activity of the elderly person during a dialogue and provide a related topic. For example, it provides a topic in which the elderly person has shown interest on social media. It can also provide a topic related to a person whom the elderly person follows on social media. Furthermore, it can provide a topic related to an article shared by the elderly person on social media. Thereby, a related topic based on the social media activity of the elderly person can be provided. Specifically, the present dialogue unit uses a Graph Neural Network (GNN) to analyze a social graph (friend relationships, follow relationships) and an interaction graph (likes, comments) of the user, and predicts a topic that is trending within a community to which the user belongs. The present dialogue unit provides a conversation that makes the user feel a “connection with society” by proactively incorporating into the dialogue news or photos that the user has not yet seen but friends are talking about. In addition, when there is a post from a grandchild or a child, the dialogue unit notifies the user of the post as a highest-priority topic, playing a role as a hub that revitalizes communication among family members.

The analysis unit can estimate an emotion of the elderly person and adjust accuracy of analysis based on the estimated emotion of the elderly person. For example, when the elderly person is relaxed, detailed analysis is performed. Also, when the elderly person feels stress, simplified analysis can be performed. Furthermore, when the elderly person is excited, analysis emphasizing changes in emotion can be performed. Thereby, the accuracy of analysis can be adjusted according to the emotion of the elderly person. Specifically, the present analysis unit has a control mechanism that makes a sampling rate of input data and a granularity of feature extraction variable. When the user is “relaxed” and high-quality data acquisition can be expected, the present analysis unit executes a high-precision mode in which voice data is processed at high resolution (e.g., 48 kHz/24 bit) to analyze even minute voice tremors (jitter, shimmer). On the other hand, when the user is in a “stressed” state and uncooperative, data quality deteriorates, so the analysis unit switches to a low-precision/high-speed mode that uses only robust feature quantities such as fundamental frequency and speech rate. Thereby, optimal allocation of analysis resources according to data quality is performed.

The analysis unit can adjust a level of detail of analysis based on importance of dialogue content during analysis. For example, detailed analysis is performed for important dialogue content. Also, simplified analysis can be performed for daily dialogue content. Furthermore, particularly detailed analysis can be performed for dialogue content regarding the health of the elderly person. Thereby, the level of detail of analysis can be adjusted according to the importance of the dialogue content. Specifically, the present analysis unit arranges a screening model that assigns a “medical relevance score” to dialogue text in a preceding stage. For an utterance with a high score (e.g., “my chest hurts”, “I forgot my medicine”), a deep natural language processing pipeline including dependency parsing and semantic analysis is applied to completely extract 5W1H information and save it as structured data. On the other hand, for an utterance with a low score (e.g., “it's nice weather”), only simple sentiment polarity determination is performed to save log capacity. This hierarchical analysis processing achieves both reduction of storage costs and improvement of searchability of important information.

The analysis unit can apply a different analysis algorithm according to a category of dialogue content during analysis. For example, a health analysis algorithm is applied to dialogue content regarding health. Also, a hobby analysis algorithm can be applied to dialogue content regarding hobbies. Furthermore, a news analysis algorithm can be applied to dialogue content regarding news. Thereby, an analysis algorithm according to the category of the dialogue content can be applied. Specifically, the present analysis unit functions as a platform that manages a group of plug-in type algorithm modules (health analysis plug-in, hobby analysis plug-in, etc.). A dynamic linking mechanism is adopted that loads only the corresponding plug-in into a memory and executes it based on a category classification result of the dialogue content. For example, in a “health” category, a symptom extraction algorithm using a medical dictionary is executed, and in a “news” category, a proper noun extraction and fact-checking algorithm is executed. This modularized design facilitates addition of new categories and analysis technologies, promoting evolution of the system.

The flow of processing of Example of the Embodiment will be briefly described below. Specifically, a data processing flow in the present system defines a series of processes from sensor input to actuator output as an event-driven state machine, and achieves both real-time performance and high throughput by executing asynchronous parallel processing in each state transition. Each step shown below is a logical processing unit, and in actual implementation, may be executed in an overlapping manner by pipeline processing.

Step 1: The dialogue unit receives an input from the elderly person. The input from the elderly person includes, for example, voice input, text input, and the like. When the elderly person speaks, the dialogue unit receives the content thereof.

Step 2: The analysis unit analyzes the input received by the dialogue unit. The analysis includes, for example, natural language processing, emotion analysis, and the like. The analysis unit analyzes utterance content, tone of voice, facial expression, etc. of the elderly person, and grasps the cognitive function and emotional state of the elderly person.

Step 3: The monitoring unit monitors the cognitive function and the behavioral pattern of the elderly person based on information analyzed by the analysis unit. The monitoring includes, for example, recording of behavioral patterns, frequency of monitoring, and the like. The monitoring unit observes the behavior of the elderly person and records changes in daily life.

Step 4: The health management unit performs individual health management based on information obtained by the monitoring unit. The health management includes, for example, collection of health data, frequency of management, and the like. When the health management unit detects a decline in the cognitive function of the elderly person, it proposes appropriate measures.

Step 5: The prediction unit performs progression prediction of dementia based on the information obtained by the monitoring unit. The prediction includes, for example, a prediction algorithm, data to be used, and the like. The prediction unit analyzes the behavioral pattern of the elderly person and predicts the progression of dementia. Specifically, in Step 1, the dialogue unit performs beamforming processing and noise canceling processing on multi-channel audio signals from a microphone array, extracts clear audio data, and stores it in a buffer. In Step 2, the analysis unit executes inference processing using a multimodal deep learning model on the audio data read from the buffer and image data acquired in synchronization, and generates an analysis result object (JSON format) including converted text utterance content, an emotion vector, and a cognitive function score. In Step 3, the monitoring unit stores the generated analysis result object in a time-series database, performs difference comparison with past history data, and sets an alert flag for behavioral change using an anomaly detection algorithm. In Step 4, when the alert flag is detected, the health management unit determines an optimal intervention plan (change of dialogue scenario, notification to family, etc.) using an inference engine, and issues a control command to the dialogue unit. In Step 5, the prediction unit performs prognosis prediction simulation by batch processing using accumulated long-term data, updates a future risk assessment report, and publishes it to a portal for medical professionals. These series of processes are executed in high-speed cooperation among the modules while maintaining data consistency.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of a plurality of elements including the above-described dialogue unit, analysis unit, monitoring unit, health management unit, and prediction unit is implemented by, for example, at least one of a smart device 14 and a data processing apparatus 12. For example, the dialogue unit is implemented by a control unit 46A of the smart device 14 and receives a voice input from the elderly person. The analysis unit is implemented by, for example, a specific processing unit 290 of the data processing apparatus 12 and analyzes the received input. The monitoring unit is implemented by, for example, the specific processing unit 290 of the data processing apparatus 12 and monitors the cognitive function and the behavioral pattern of the elderly person based on the analyzed information. The health management unit is implemented by, for example, the specific processing unit 290 of the data processing apparatus 12 and performs individual health management based on the information obtained by the monitoring unit. The prediction unit is implemented by, for example, the specific processing unit 290 of the data processing apparatus 12 and performs progression prediction of dementia based on the information obtained by the monitoring unit. The correspondence relationship between each unit and the apparatus or the control unit is not limited to the above-described example, and various modifications are possible.

### Second Embodiment

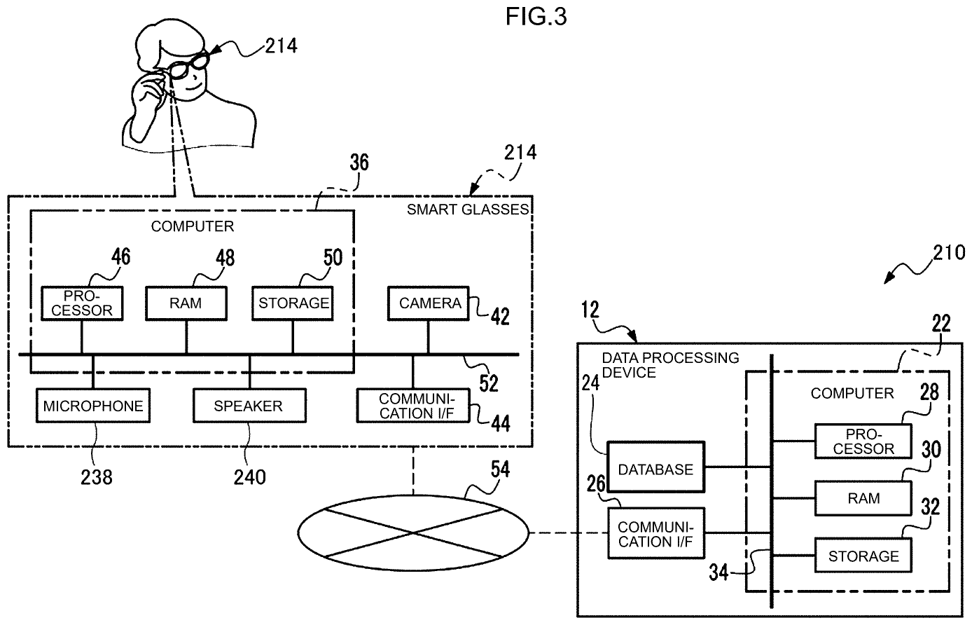

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

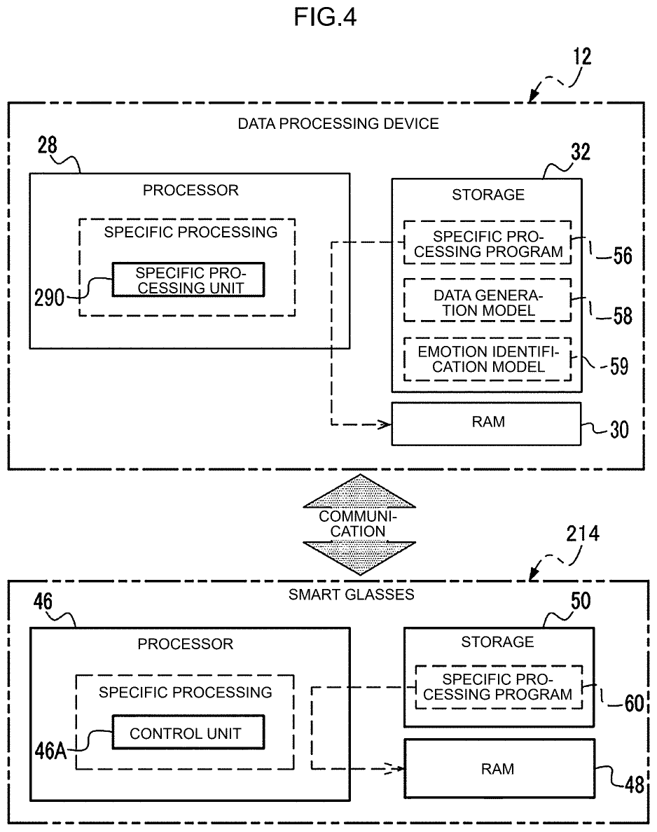

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of a plurality of elements including the above-described dialogue unit, analysis unit, monitoring unit, health management unit, and prediction unit is implemented by, for example, at least one of smart glasses 214 and a data processing apparatus 12. For example, the dialogue unit is implemented by a control unit 46A of the smart glasses 214 and receives a voice input from the elderly person. The analysis unit is implemented by, for example, a specific processing unit 290 of the data processing apparatus 12 and analyzes the received input. The monitoring unit is implemented by, for example, the specific processing unit 290 of the data processing apparatus 12 and monitors the cognitive function and the behavioral pattern of the elderly person based on the analyzed information. The health management unit is implemented by, for example, the specific processing unit 290 of the data processing apparatus 12 and performs individual health management based on the information obtained by the monitoring unit. The prediction unit is implemented by, for example, the specific processing unit 290 of the data processing apparatus 12 and performs progression prediction of dementia based on the information obtained by the monitoring unit. The correspondence relationship between each unit and the apparatus or the control unit is not limited to the above-described example, and various modifications are possible.

### Third Embodiment

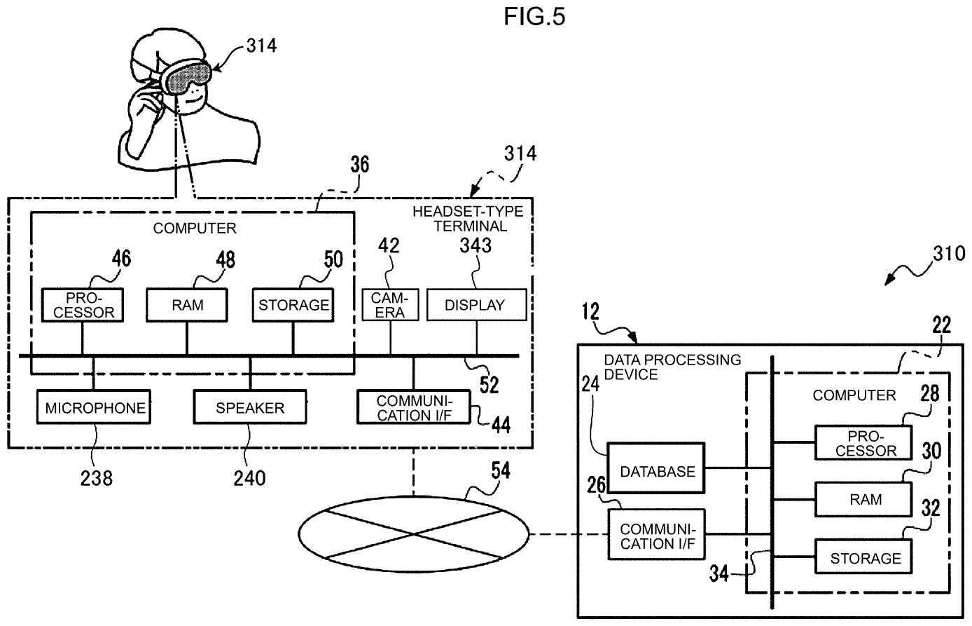

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

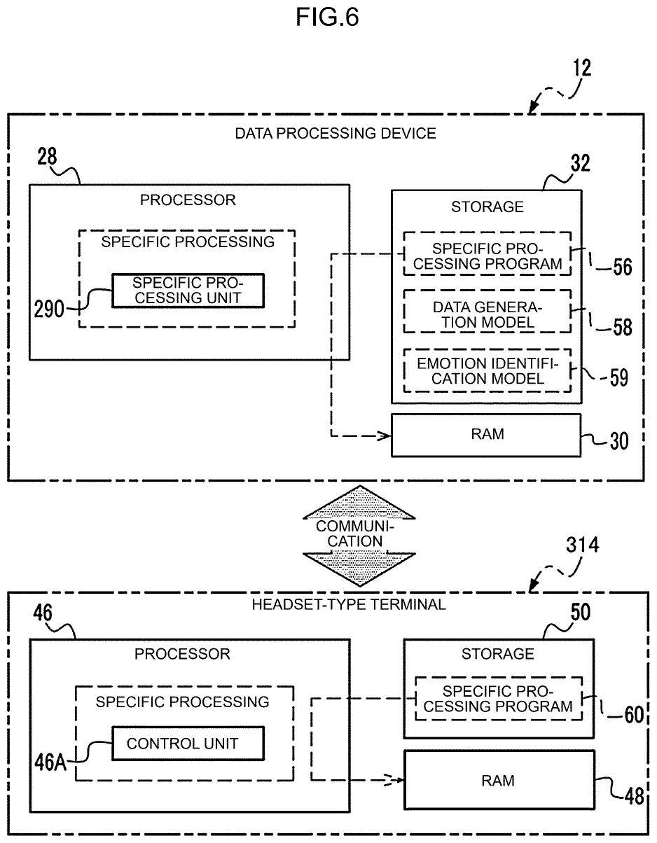

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of a plurality of elements including the above-described dialogue unit, analysis unit, monitoring unit, health management unit, and prediction unit is implemented by, for example, at least one of a headset-type terminal 314 and a data processing apparatus 12. For example, the dialogue unit is implemented by a control unit 46A of the headset-type terminal 314 and receives a voice input from the elderly person. The analysis unit is implemented by, for example, a specific processing unit 290 of the data processing apparatus 12 and analyzes the received input. The monitoring unit is implemented by, for example, the specific processing unit 290 of the data processing apparatus 12 and monitors the cognitive function and the behavioral pattern of the elderly person based on the analyzed information. The health management unit is implemented by, for example, the specific processing unit 290 of the data processing apparatus 12 and performs individual health management based on the information obtained by the monitoring unit. The prediction unit is implemented by, for example, the specific processing unit 290 of the data processing apparatus 12 and performs progression prediction of dementia based on the information obtained by the monitoring unit. The correspondence relationship between each unit and the apparatus or the control unit is not limited to the above-described example, and various modifications are possible.

### Fourth Embodiment

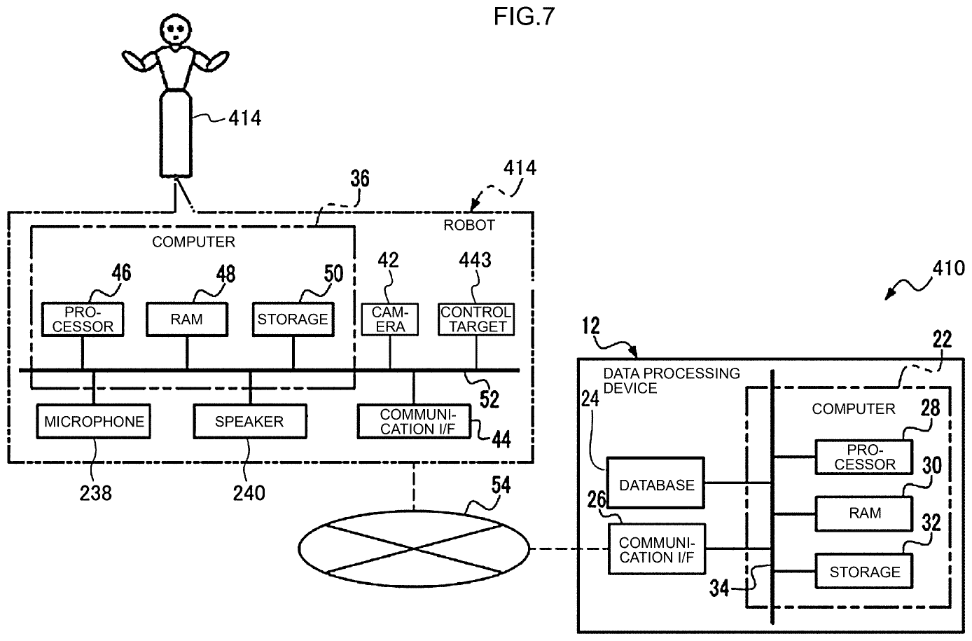

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

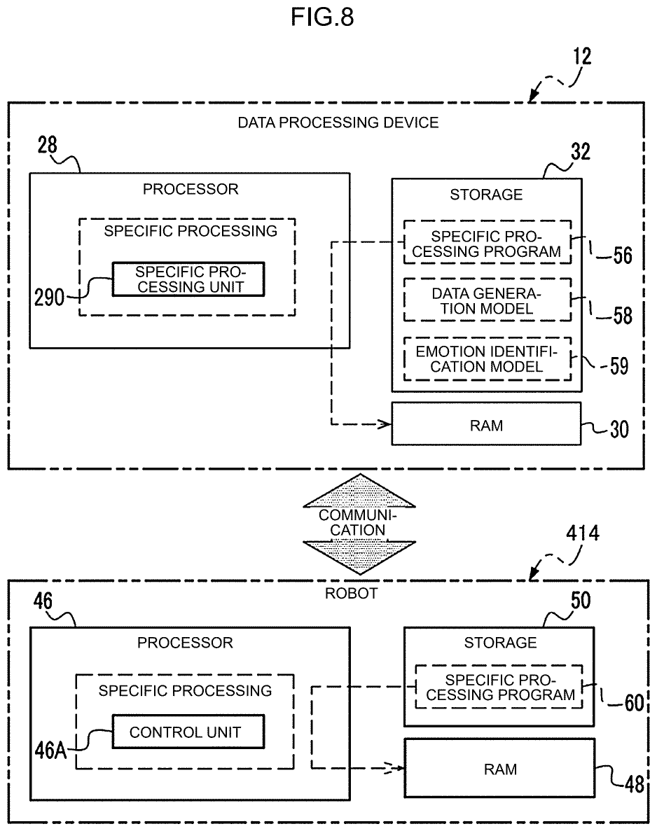

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of a plurality of elements including the above-described dialogue unit, analysis unit, monitoring unit, health management unit, and prediction unit is implemented by, for example, at least one of a robot 414 and a data processing apparatus 12. For example, the dialogue unit is implemented by a control unit 46A of the robot 414 and receives a voice input from the elderly person. The analysis unit is implemented by, for example, a specific processing unit 290 of the data processing apparatus 12 and analyzes the received input. The monitoring unit is implemented by, for example, the specific processing unit 290 of the data processing apparatus 12 and monitors the cognitive function and the behavioral pattern of the elderly person based on the analyzed information. The health management unit is implemented by, for example, the specific processing unit 290 of the data processing apparatus 12 and performs individual health management based on the information obtained by the monitoring unit. The prediction unit is implemented by, for example, the specific processing unit 290 of the data processing apparatus 12 and performs progression prediction of dementia based on the information obtained by the monitoring unit. The correspondence relationship between each unit and the apparatus or the control unit is not limited to the above-described example, and various modifications are possible.

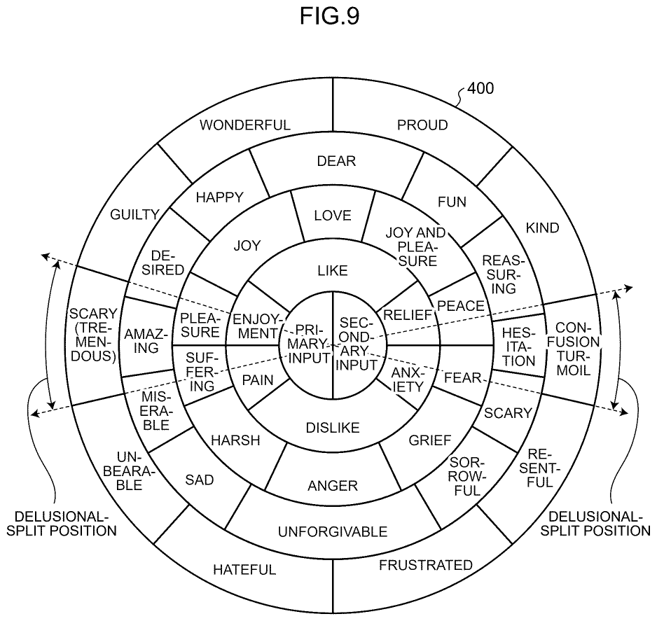

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

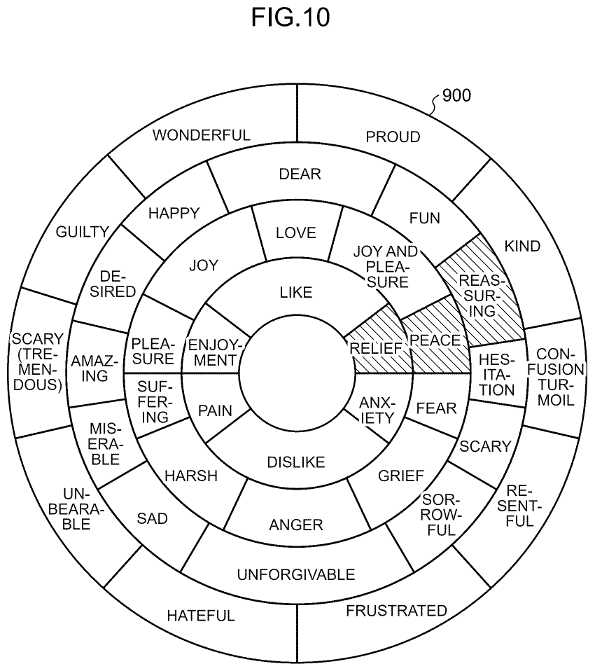

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a dialogue unit configured to receive an input from an elderly person; an analysis unit configured to analyze the input received by the dialogue unit; a monitoring unit configured to monitor a cognitive function and a behavioral pattern of the elderly person based on information analyzed by the analysis unit; a health management unit configured to perform individual health management based on information obtained by the monitoring unit; and a prediction unit configured to perform progression prediction of dementia based on the information obtained by the monitoring unit.
(Supplementary Note 2) The system according to Supplementary Note 1, wherein the dialogue unit is configured to estimate an emotion of the elderly person and adjust content or a tone of a dialogue based on the estimated emotion of the elderly person.
(Supplementary Note 3) The system according to Supplementary Note 1, wherein the dialogue unit is configured to analyze a past dialogue history of the elderly person and select an appropriate dialogue method.
(Supplementary Note 4) The system according to Supplementary Note 1, wherein the dialogue unit is configured to filter dialogue content based on a current health condition or an interest of the elderly person during a dialogue.
(Supplementary Note 5) The system according to Supplementary Note 1, wherein the dialogue unit is configured to estimate an emotion of the elderly person and adjust a frequency of a dialogue based on the estimated emotion of the elderly person.
(Supplementary Note 6) The system according to Supplementary Note 1, wherein the dialogue unit is configured to preferentially take up a highly relevant topic based on geographical location information of the elderly person during a dialogue.
(Supplementary Note 7) The system according to Supplementary Note 1, wherein the dialogue unit is configured to analyze social media activity of the elderly person during a dialogue and provide a related topic.
(Supplementary Note 8) The system according to Supplementary Note 1, wherein the analysis unit is configured to estimate an emotion of the elderly person and adjust accuracy of analysis based on the estimated emotion of the elderly person.
(Supplementary Note 9) The system according to Supplementary Note 1, wherein the analysis unit is configured to adjust a level of detail of analysis based on importance of dialogue content during analysis.
(Supplementary Note 10) The system according to Supplementary Note 1, wherein the analysis unit is configured to apply a different analysis algorithm according to a category of dialogue content during analysis.
(Supplementary Note 11) The system according to Supplementary Note 1, wherein the analysis unit is configured to estimate an emotion of the elderly person and adjust a display method of an analysis result based on the estimated emotion of the elderly person.
(Supplementary Note 12) The system according to Supplementary Note 1, wherein the analysis unit is configured to determine a priority of analysis based on a time zone of a dialogue during analysis.
(Supplementary Note 13) The system according to Supplementary Note 1, wherein the analysis unit is configured to adjust an order of analysis based on relevance of dialogue content during analysis.
(Supplementary Note 14) The system according to Supplementary Note 1, wherein the monitoring unit is configured to estimate an emotion of the elderly person and adjust a criterion for monitoring based on the estimated emotion of the elderly person.
(Supplementary Note 15) The system according to Supplementary Note 1, wherein the monitoring unit is configured to record a change in a behavioral pattern of the elderly person in real time during monitoring.
(Supplementary Note 16) The system according to Supplementary Note 1, wherein the monitoring unit is configured to improve accuracy of monitoring based on a change in a living environment of the elderly person during monitoring.
(Supplementary Note 17) The system according to Supplementary Note 1, wherein the monitoring unit is configured to estimate an emotion of the elderly person and adjust a display method of a monitoring result based on the estimated emotion of the elderly person.
(Supplementary Note 18) The system according to Supplementary Note 1, wherein the monitoring unit is configured to perform monitoring in consideration of a geographical distribution of the elderly person during monitoring.
(Supplementary Note 19) The system according to Supplementary Note 1, wherein the monitoring unit is configured to improve accuracy of monitoring by referring to related literature of the elderly person during monitoring.
(Supplementary Note 20) The system according to Supplementary Note 1, wherein the health management unit is configured to estimate an emotion of the elderly person and adjust a method of health management based on the estimated emotion of the elderly person.
(Supplementary Note 21) The system according to Supplementary Note 1, wherein the health management unit is configured to select an optimal health management method by referring to past health data of the elderly person during health management.
(Supplementary Note 22) The system according to Supplementary Note 1, wherein the health management unit is configured to customize a means for health management based on a current living situation of the elderly person during health management.
(Supplementary Note 23) The system according to Supplementary Note 1, wherein the health management unit is configured to estimate an emotion of the elderly person and determine a priority of health management based on the estimated emotion of the elderly person.
(Supplementary Note 24) The system according to Supplementary Note 1, wherein the health management unit is configured to select an optimal health management method in consideration of geographical location information of the elderly person during health management.
(Supplementary Note 25) The system according to Supplementary Note 1, wherein the health management unit is configured to analyze social media activity of the elderly person and propose a means for health management during health management.
(Supplementary Note 26) The system according to Supplementary Note 1, wherein the prediction unit is configured to estimate an emotion of the elderly person and adjust a method of progression prediction of dementia based on the estimated emotion of the elderly person.
(Supplementary Note 27) The system according to Supplementary Note 1, wherein the prediction unit is configured to select an optimal prediction method by referring to past cognitive function data of the elderly person during prediction.
(Supplementary Note 28) The system according to Supplementary Note 1, wherein the prediction unit is configured to customize a means for progression prediction of dementia based on a current living situation of the elderly person during prediction.
(Supplementary Note 29) The system according to Supplementary Note 1, wherein the prediction unit is configured to estimate an emotion of the elderly person and determine a priority of progression prediction of dementia based on the estimated emotion of the elderly person.
(Supplementary Note 30) The system according to Supplementary Note 1, wherein the prediction unit is configured to select an optimal prediction method in consideration of geographical location information of the elderly person during prediction.
(Supplementary Note 31) The system according to Supplementary Note 1, wherein the prediction unit is configured to analyze social media activity of the elderly person and propose a means for progression prediction of dementia during prediction.

What is claimed is:

1. A system comprising:
circuitry configured to:
receive, from a client terminal via a packet-switched network, input data comprising at least one of text data, voice data, or image data;
generate analysis data by inputting the input data into a data generation model, the analysis data comprising a classification label and a feature vector;
generate time-series deviation data by inputting the analysis data into a time-series analysis model comprising at least one of a recurrent neural network or a long short-term memory network, the time-series deviation data representing a deviation of a current feature vector from a reference feature vector calculated from feature vectors accumulated over a plurality of sessions;
generate, based on the time-series deviation data, first inference data by inputting the time-series deviation data and the classification label into an inference engine; and
generate second inference data by inputting the time-series deviation data into a prediction model comprising at least one of a generative adversarial network or a variational autoencoder, the second inference data comprising a predicted future value of the deviation.

2. The system according to claim 1, wherein the circuitry is further configured to estimate an emotion of a user associated with the input data by inputting at least one of the text data, the voice data, or the image data into an emotion identification model, and to adjust content or a tone of response data transmitted to the client terminal based on the estimated emotion.

3. The system according to claim 1, wherein the circuitry is further configured to analyze a past session history associated with the user by vectorizing past session logs and storing the vectorized past session logs in a vector database, and to select a session method by retrieving, from the vector database, a past session segment having a cosine similarity to a current session context that exceeds a similarity threshold.

4. The system according to claim 1, wherein the circuitry is further configured to filter response candidates generated by the data generation model based on a user profile vector, the user profile vector comprising a multidimensional feature vector representing at least one of a current condition or an interest of the user, and to calculate a fitness score for each response candidate by inner product calculation between a topic vector of the response candidate and the user profile vector.

5. The system according to claim 1, wherein the circuitry is further configured to estimate an emotion of a user associated with the input data, and to adjust a frequency of transmitting the response data to the client terminal based on the estimated emotion by optimizing a policy network of a reinforcement learning model in which the estimated emotion is a state variable and a transmission timing is an action variable.

6. The system according to claim 1, wherein the circuitry is further configured to receive geographic location information from the client terminal, and to select a topic for inclusion in the response data based on the geographic location information by collecting location-based information within a predetermined radius of the geographic location information.

7. The system according to claim 1, wherein the circuitry is further configured to analyze social media activity data associated with the user by applying a graph neural network to a social graph and an interaction graph derived from the social media activity data, and to select a topic for inclusion in the response data based on a trending topic predicted from the social graph.

8. The system according to claim 1, wherein the circuitry is further configured to estimate an emotion of a user associated with the input data, and to dynamically switch a scale of the data generation model used to generate the analysis data based on the estimated emotion, such that when the estimated emotion indicates a relaxed state, the circuitry selects a large-scale model for high-precision analysis, and when the estimated emotion indicates a stressed state, the circuitry selects a lightweight model for reduced-latency analysis.

9. The system according to claim 1, wherein the circuitry is further configured to determine an importance score for the input data by applying an importance determination model using an attention mechanism, and to apply a full natural language understanding process comprising syntax analysis, semantic role labeling, and coreference resolution when the importance score exceeds a predetermined importance threshold, and to apply a lightweight keyword extraction process when the importance score does not exceed the predetermined importance threshold.

10. The system according to claim 1, wherein the circuitry is further configured to classify the input data into a category using a multi-class classifier comprising a neural network having a softmax layer, and to select a domain-specific analysis model from a plurality of domain-specific analysis models based on the category, the plurality of domain-specific analysis models comprising at least one of a named entity recognition model or an emotion analysis model.

11. The system according to claim 1, wherein the circuitry is further configured to estimate an emotion of a user associated with the input data, and to adjust a display format of the analysis data transmitted to the client terminal based on the estimated emotion, such that when the estimated emotion indicates nervousness, the circuitry generates the analysis data in a simplified format with an enlarged font size, and when the estimated emotion indicates relaxation, the circuitry generates the analysis data in a detailed format comprising a graph and an advice sentence.

12. The system according to claim 1, wherein the circuitry is further configured to determine a priority of analyzing the input data based on a timestamp associated with the input data by assigning the input data to a high-priority processing queue when the timestamp corresponds to a first time zone and assigning the input data to a low-priority processing queue for batch processing when the timestamp corresponds to a second time zone.

13. The system according to claim 1, wherein the circuitry is further configured to estimate an emotion of a user associated with the input data, and to dynamically adjust a determination threshold in an anomaly detection algorithm used to generate the time-series deviation data based on the estimated emotion, such that when the estimated emotion indicates stress, the circuitry relaxes the determination threshold, and when the estimated emotion indicates excitement, the circuitry tightens the determination threshold.

14. The system according to claim 1, wherein the circuitry is further configured to record a change in a behavioral pattern associated with the user in real time by writing time-stamped behavioral data to a database, and to generate an alert signal when the change exceeds a predetermined change threshold.

15. The system according to claim 1, wherein the circuitry is further configured to generate the first inference data by reading time-series data accumulated as a personal record of the user from a database, modeling an individual trend using the time-series analysis model, and recommending an intervention method that was effective in a past period based on the individual trend.

16. The system according to claim 1, wherein the circuitry is further configured to estimate an emotion of a user associated with the input data, and to generate the second inference data by adding an emotion state vector derived from the estimated emotion as an input feature to the prediction model, and applying a correction coefficient to a predicted score based on the emotion state vector to estimate a baseline value.

17. The system according to claim 1, wherein the circuitry is further configured to generate the second inference data by performing linguistic analysis on social media post data associated with the user to quantify at least one of vocabulary diversity, grammatical complexity, or a change in posting frequency, and integrating a result of the linguistic analysis with the time-series deviation data in the prediction model.

18. A system comprising:
a communication interface connected to a packet-switched network;
a processor;
a random access memory;
a non-volatile storage storing a data generation model, an emotion identification model, and a program;
a database; and
circuitry configured to execute the program, the circuitry being configured to:
receive, via the communication interface from a client terminal connected to the packet-switched network, input data comprising at least one of text data converted from voice data acquired by a microphone of the client terminal, the voice data, or image data acquired by a camera of the client terminal;
generate analysis data by inputting the input data into the data generation model stored in the non-volatile storage, the analysis data comprising a classification label and a feature vector, the data generation model comprising a neural network obtained by deep learning;
estimate an emotion of a user associated with the input data by inputting at least one of the text data, the voice data, or the image data into the emotion identification model stored in the non-volatile storage;
store the analysis data and the estimated emotion in the database;
generate time-series deviation data by inputting the analysis data into a time-series analysis model comprising at least one of a recurrent neural network or a long short-term memory network, the time-series deviation data representing a deviation of a current feature vector from a reference feature vector calculated from feature vectors accumulated over a plurality of sessions and stored in the database;
generate, based on the time-series deviation data, first inference data by inputting the time-series deviation data and the classification label into an inference engine;
generate second inference data by inputting the time-series deviation data into a prediction model comprising at least one of a generative adversarial network or a variational autoencoder, the second inference data comprising a predicted future value of the deviation; and
transmit, via the communication interface to the client terminal, response data based on at least one of the first inference data or the second inference data.

19. The system according to claim 18, wherein the client terminal comprises at least one of a smart device comprising a touch panel and a display, smart glasses comprising a microphone and a speaker, a headset-type terminal comprising a microphone, a speaker, a camera, and a display, or a robot comprising a microphone, a speaker, a camera, and a control target including a motor for driving an articulated mechanism.

20. A method performed by circuitry of a system, the method comprising:
receiving, from a client terminal via a packet-switched network, input data comprising at least one of text data, voice data, or image data;
generating analysis data by inputting the input data into a data generation model, the analysis data comprising a classification label and a feature vector;
generating time-series deviation data by inputting the analysis data into a time-series analysis model comprising at least one of a recurrent neural network or a long short-term memory network, the time-series deviation data representing a deviation of a current feature vector from a reference feature vector calculated from feature vectors accumulated over a plurality of sessions;
generating, based on the time-series deviation data, first inference data by inputting the time-series deviation data and the classification label into an inference engine; and
generating second inference data by inputting the time-series deviation data into a prediction model comprising at least one of a generative adversarial network or a variational autoencoder, the second inference data comprising a predicted future value of the deviation.
